(19) United States
(12) Patent Application Publication
(10) Pub. No.: US 2018/0327842 A1
(43) Pub. Date: Nov. 15, 2018

(54) METHOD FOR IDENTIFYING A GREATER RISK FOR DEVELOPING BRONCHOPULMONARY DYSPLASIA

(71) Applicant: Meribank Biotech Co., Ltd., New Taipei City (TW)

(72) Inventors: Chang-Yo HSUAN, Taipei City (TW); Willie LIN, Taipei City (TW); Wei-Ting LIU, Taipei City (TW); Meng-Hua LEE, Taipei City (TW); Ting-Ting TSENG, Taipei City (TW)

(73) Assignee: Meribank Biotech Co., Ltd., New Taipei City (TW)

(21) Appl. No.: 15/592,874

(22) Filed: May 11, 2017

(51) Int. Cl. C12Q 1/68

(52) U.S. Cl. CPC C12Q 1/6883; C12Q 2600/156; C12Q 2600/158

(57) ABSTRACT

Disclosed is a method of identifying a greater risk for developing bronchopulmonary dysplasia (BPD) in a preterm infant. The method comprises obtaining a genomic DNA sample from the preterm infant's mother, identifying the nucleotide of rs2280789 SNP in the RANTES gene and the nucleotide of rs1800566 SNP in the NQO1 gene, and determining the preterm infant as being at risk of developing BPD when the genotype of rs2280789 SNP carries C nucleotide and the genotype of rs1800566 SNP carries T nucleotide.

### FIELD OF THE INVENTION

The present invention pertains to a method for identifying a greater risk for developing bronchopulmonary dysplasia (BPD) of preterm birth.

### BACKGROUND OF THE INVENTION

Preterm birth (PTB), or birth before 37 weeks of gestation period, is the major cause of neonatal mortality and morbidity worldwide. Approximately 70% of the neonatal deaths are due to preterm delivery.

Bronchopulmonary dysplasia (BPD), a common chronic inflammatory lung disease of very-low-birth-weight (VLBW) preterm infants, is associated with arrested lung development and treatment of supplemental oxygen [1]. Due to the influences of long-term oxygen therapy and mechanical ventilation, many of these preterm infants consequently acquire different types of problems, such as highly reactive airway diseases, recurrent lower respiratory tract infections, abrupt alveolar development, growth retardation, and feeding difficulties [2, 3]. While early detection of BPD is crucial to prevent chronic symptoms and complications later in life, diagnosis and prevention of this disease remains challenging due to the lack of good biomarkers for identification of infants at risk [1]. It has been reported that interleukin-8 (IL-8) and C-reactive protein (CRP), two recently identified preterm biomarkers, can also be biomarkers for BPD [4, 5].

There is still an urgent need for novel and efficient biomarkers for BPD.

### BRIEF SUMMARY OF THE INVENTION

The present invention provides a method for identifying a greater risk for developing bronchopulmonary dysplasia (BPD) of a preterm infant, comprising obtaining a genomic DNA sample from the preterm infant's mother, identifying the nucleotide of rs2280789 SNP in the RANTES gene and the nucleotide of rs1800566 SNP in the NQO1 gene, and determining the preterm infant as being at risk of developing BPD when the genotype of rs2280789 SNP carries C nucleotide and the genotype of rs1800566 SNP carries T nucleotide.

In certain embodiments of the present invention, the determination is further based on the birth weight of the preterm infant, wherein a lower birth weight indicates an increased risk of developing BPD.

In certain embodiments of the present invention, the determination is further based on a specific condition in the preterm infant's mother selected from the group consisting of an amniotic band syndrome, a placenta rupture during delivery, a shortness of umbilical cord, and a combination thereof, where a higher number of the specific conditions indicates an increased risk of developing BPD.

According to the present invention, for rs2280789 SNP, the genotype CC indicates a higher risk of developing BPD than the genotype TT.

According to the present invention, for rs1800566 SNP, the genotype TT indicates a higher risk of developing BPD than the genotype CC.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the BPD risk scores for 15 preterm infants assessed by a method of the present invention.

### DETAILED DESCRIPTION OF THE INVENTION

The present invention provides a method for determining the risk of bronchopulmonary dysplasia (BPD) or identifying a greater risk for developing BPD of a preterm infant. The method comprises obtaining a genomic DNA sample from the preterm infant's mother, identifying the nucleotide of rs2280789 SNP in the RANTES gene and the nucleotide of rs1800566 SNP in the NQO1 gene, and determining the preterm infant as being at risk of developing BPD when the genotype of rs2280789 SNP carries C nucleotide and the genotype of rs1800566 SNP carries T nucleotide.

The term “SNP” as used herein means a single nucleotide polymorphism which is a single nucleotide position in a nucleotide sequence for which two or more alternative alleles are present in a given population.

In certain embodiments of the present invention, the determination is further based on the birth weight of the preterm infant, wherein a lower birth weight indicates an increased risk of developing BPD. Preferably, a birth weight less than 1,000 g indicates an increased risk of developing BPD. A lower birth weight indicates a higher risk of developing BPD.

In certain embodiments of the present invention, the determination is further based on a specific condition in the preterm infant's mother selected from the group consisting of an amniotic fibrous band, a placenta rupture during delivery, a shortness of umbilical cord, and a combination thereof, where a higher number of the specific conditions indicates an increased risk of developing BPD.

According to the present invention, the shortness of umbilical cord preferably refers to a umbilical cord with a length less than 30 cm, 29.5 cm, 29 cm, 28.5 cm, or 28 cm.

According to the present invention, for rs2280789 SNP, the genotype CC indicates a higher risk of developing BPD than the genotype TC, and the genotype TC indicates a higher risk of developing BPD than the genotype TT.

According to the present invention, for rs1800566 SNP, the genotype TT indicates a higher risk of developing BPD than the genotype TC, and the genotype TC indicates a higher risk of developing BPD than the genotype CC.

In certain embodiments of the present invention, the genotype TC of rs2280789 SNP is given a higher weight with respect to a greater risk for developing BPD than the genotype TC of rs1800566 SNP.

According to the present invention, the genomic DNA sample may be derived from a tissue selected from the group consisting of blood, placenta, amniotic membrane, chorionic disk, chorionic membrane, and umbilical cord, but is not limited thereto. In one embodiment of the present invention, the blood is umbilical cord blood. In another embodiment, the blood is peripheral blood.

The present invention is further illustrated by the following examples, which are provided for the purpose of demonstration rather than limitation.

### EXAMPLES

### Example 1

Briefly, the genomic DNAs were isolated from mesenchymal stem cells derived from the placenta of 15 mothers of respective preterm infants. The RANTES gene and the NQO1 gene were amplified using PCR, and then were subjected to Sanger sequencing to identify the nucleotide of rs2280789 SNP and the nucleotide of rs1800566 SNP, respectively. In a parallel test, genomic DNAs were isolated from umbilical cord blood sample and the nucleotide of rs2280789 SNP and the nucleotide of rs1800566 SNP were identified, respectively (data not shown).

The risk scores of developing BPD were calculated according to the following rules:

(1) RANTES and NQO1 Genes SNPs

For RANTES T/C (rs2280789), the genotype TT (wild-type) is given a score of 10, the genotype TC (hetero-type) is given a score of 40, and the genotype CC (mutant type) is given a score of 50. For NQO1 T/C (rs1800566), the genotype CC (wild-type) is given a score of 20, the genotype TC (hetero-type) is given a score of 30, and the genotype TT (mutant type) is given a score of 50.

(2) Birth Weights of Infants

The scores are given as follows: ≤500 g→20; 501-600 g→16; 601-700 g→13; 701-800 g→10; 801-900 g→7; 901-1,000 g 4; ≥1,000 g→1.

(3) Specific Conditions

The specific conditions include an amniotic fibrous band, a placenta rupture during delivery, a shortness of umbilical cord (length less than 30 cm). The observation of each condition is given a score of 5.

The scores are shown in Table 1 below.

TABLE 1
Risk Scores

| | | | | Specific | |
|---|---|---|---|---|---|
| Sample | RANTES | NQO1 | Weight | condition | Total |
| name | SNP score | SNP score | score | score | score |
| TSG002* | 50 | 50 | 20 | 10 | 130 |
| TSG010* | 50 | 50 | 13 | 10 | 123 |
| CK004* | 50 | 30 | 4 | 10 | 94 |
| LCG009* | 40 | 50 | 1 | 0 | 91 |
| LCG006* | 50 | 30 | 7 | 0 | 87 |
| CK017 | 50 | 30 | 4 | 0 | 84 |
| LCG014 | 50 | 20 | 7 | 5 | 82 |
| TSG008* | 40 | 20 | 13 | 5 | 78 |
| CK005 | 40 | 30 | 7 | 0 | 77 |
| LCG012 | 40 | 30 | 7 | 0 | 77 |
| CK016 | 40 | 30 | 1 | 0 | 71 |
| CK001 | 40 | 20 | 1 | 10 | 71 |
| CK013 | 40 | 20 | 7 | 0 | 67 |
| LCG011 | 10 | 30 | 1 | 10 | 51 |
| TSG015 | 10 | 30 | 1 | 5 | 46 |

Sample names marked with “*” refer to samples from BPD infants' mothers.

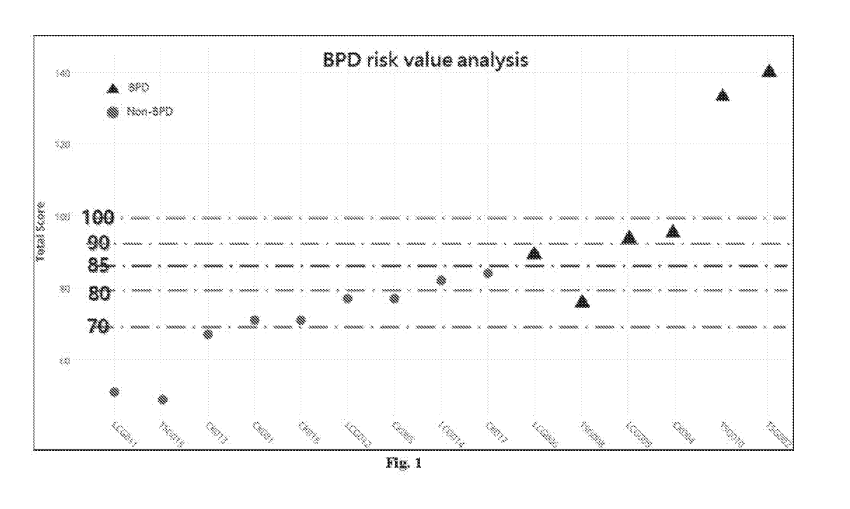

The results on the total scores are also shown in FIG. 1. As can be seen in FIG. 1, different cutoff scores (e.g. 80, 85, 90, etc.) can be selected to obtain desired positive predictive values (PPVs) and/or negative predictive values (NPVs), summarized in Table 2 below.

TABLE 2
PPVs and NPVs for different cutoff scores

| | | | Diagnostic |
|---|---|---|---|
| | BPD | Non-BPD | Accuracy |
| Cut off at 70 | | | |
| Test positive (+) | 6 | 6 | PPV = 50% |
| Test negative (−) | 0 | 3 | NPV = 100% |
| Cut off at 80 | | | |
| Test positive (+) | 5 | 2 | PPV = 71% |
| Test negative (−) | 1 | 7 | NPV = 88% |
| Cut off at 85 | | | |
| Test positive (+) | 5 | 0 | PPV = 100% |
| Test negative (−) | 1 | 9 | NPV = 90% |
| Cut off at 90 | | | |
| Test positive (+) | 4 | 0 | PPV = 100% |
| Test negative (−) | 2 | 9 | NPV = 82% |
| Cut off at 100 | | | |
| Test positive (+) | 2 | 0 | PPV = 100% |
| Test negative (−) | 4 | 9 | NPV = 69% |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### REFERENCES

[1] Rivera L, Siddaiah R, Oji-Mmuo C, Silveyra G R, Silveyra P. Biomarkers for Bronchopulmonary Dysplasia in the Preterm Infant. Frontiers in pediatrics 2016; 4:33.
[2] Yang Y, Qiu J, Kan Q, Zhou X G, Zhou X Y. MicroRNA expression profiling studies on bronchopulmonary dysplasia: a systematic review and meta-analysis. Genetics and molecular research: GMR 2013; 12:5195-206.
[3] Maitre N L, Ballard R A, Ellenberg J H, Davis S D, Greenberg J M, Hamvas A, et al. Respiratory consequences of prematurity: evolution of a diagnosis and development of a comprehensive approach. Journal of perinatology: official journal of the California Perinatal Association 2015; 35:313-21.
[4] Paananen R, Husa A K, Vuolteenaho R, Herva R, Kaukola T, Hallman M. Blood cytokines during the perinatal period in very preterm infants: relationship of inflammatory response and bronchopulmonary dysplasia. The Journal of pediatrics 2009; 154:39-43 e3.
[5] Ambalavanan N, Carlo W A, D'Angio C T, McDonald S A, Das A, Schendel D, et al. Cytokines associated with bronchopulmonary dysplasia or death in extremely low birth weight infants. Pediatrics 2009; 123:1132-41.

1. A method of identifying a greater risk for developing bronchopulmonary dysplasia (BPD) in a preterm infant, comprising:
obtaining a genomic DNA sample from the preterm infant's mother;
identifying the nucleotide of rs2280789 SNP in the RANTES gene and the nucleotide of rs1800566 SNP in the NQO1 gene; and
determining the preterm infant as being at risk of developing BPD when the genotype of rs2280789 SNP carries C nucleotide and the genotype of rs1800566 SNP carries T nucleotide.

2. The method of claim 1, wherein the determination is further based on the birth weight of the preterm infant, wherein a lower birth weight indicates an increased risk of developing BPD.

3. The method of claim 1, wherein the determination is further based on a specific condition in the preterm infant's mother selected from the group consisting of an amniotic fibrous band, a placenta rupture during delivery, a shortness of umbilical cord, and a combination thereof, where a higher number of the specific conditions indicates an increased risk of developing BPD.

4. The method of claim 3, wherein the shortness of umbilical cord is a specific condition in the preterm infant's mother where the length of umbilical cord is less than 30 cm.

5. The method of claim 1, wherein for rs2280789 SNP the genotype CC indicates a higher risk of developing BPD than the genotype TT.

6. The method of claim 1, wherein for rs1800566 SNP the genotype TT indicates a higher risk of developing BPD than the genotype CC.

7. The method of claim 1, wherein the genomic DNA sample is derived from a tissue selected from the group consisting of blood, placenta, amniotic membrane, chorionic disk, chorionic membrane, and umbilical cord.

8. The method of claim 2, wherein the determination is further based on a specific condition in the preterm infant's mother selected from the group consisting of an amniotic fibrous band, a placenta rupture during delivery, a shortness of umbilical cord, and a combination thereof, where a higher number of the specific conditions indicates an increased risk of developing BPD.

9. The method of claim 8, wherein the shortness of umbilical cord is a specific condition in the preterm infant's mother where the length of umbilical cord is less than 30 cm.
